(19) United States
(12) Patent Application Publication
(10) Pub. No.: US 2024/0424053 A1
(43) Pub. Date: Dec. 26, 2024

(54) METHODS AND COMPOSITIONS FOR PROVIDING MYOCARDIAL PROTECTION AND TREATING MYOCARDIAL STRESS AND FIBROSIS

(71) Applicant: Tufts Medical Center, Inc., Boston, MA (US)

(72) Inventors: Athan KULIOPULOS, Winchester, MA (US); Lidija COVIC, Lexington, MA (US)

(21) Appl. No.: 18/708,292

(22) PCT Filed: Nov. 9, 2022
(86) PCT No.: PCT/US2022/049413

§ 371 Date: May 8, 2024

Related U.S. Application Data
(60) Provisional application No. 63/277,276, filed on Nov. 9, 2021.

(51) Int. Cl. A61K 38/08; A61K 9/00; A61K 31/165; A61K 31/277; A61K 31/351; A61K 31/401; A61K 31/4178; A61K 31/4184; A61K 31/4422; A61K 31/585; A61K 31/7042; A61K 31/7048; A61P 9/10

(52) U.S. Cl. CPC A61K 38/08; A61K 9/0019; A61K 31/165; A61K 31/277; A61K 31/351; A61K 31/401; A61K 31/4178; A61K 31/4184; A61K 31/4422; A61K 31/585; A61K 31/7042; A61K 31/7048; A61P 9/10

(57) ABSTRACT

The invention provides methods and compositions for providing myocardial protection and for treatment of myocardial stress and fibrosis, which include the administration of PZ-128.

### STATEMENT OF GOVERNMENT SUPPORT

This invention was made with government support under grant HL136485 awarded by the National Institutes of Health. The government has certain rights in the invention.

### FIELD OF THE INVENTION

The invention is in the field of cardiovascular medicine.

### BACKGROUND

PAR1 is a member of the protease-activated receptor family that is widely expressed in human cells, including on platelets, immune cells, endothelial cells, and a number of other tissues (Ossovskaya et al., Physiol. Rev. 84(2):579-621, 2004; Koukos et al., IUBMB Life 63(6):412-418, 2011). PAR1 has emerged as a promising target for therapeutic intervention in cardiovascular diseases, including atherothrombotic disease and acute coronary syndromes (Zhang et al., Circulation 126(1):83-91, 2012; Ahn et al., Curr. Pharm. Des. 9(28):2349-2365, 2003; Smith et al., Platelets 16(6):340-345, 2005; Wiviott et al., Circulation 123(17):1854-1863, 2011; Trivedi et al., Cell 137(2):332-343, 2009).

PAR1 modulating pepducins were identified in early PAR1 pepducin screens (Covic et al., Proc. Natl. Acad. Sci. U.S.A. 99(2):643-648, 2002) that have since demonstrated therapeutic potential in a variety of diseases. P1 pal-7, a 7mer palmitoylated pepducin also known as PZ-128 and P1 pal-12, a 12mer pepducin, both based on the 3 loop of the receptor are full PAR1 antagonists that have been shown to inhibit platelet aggregation, arterial thrombosis, atherosclerosis, inflammation, pulmonary fibrosis, tumorigenesis, metastasis, and angiogenesis, leading to significant efficacy in a variety of disease models (Covic et al., Nat. Med. 8(10):1161-1165, 2002; Wielders et al., J. Thromb. Haemost. 5 (3):571-576, 2007; Zhang et al., Circulation 126(1):83-91, 2012; Agarwal et al., Cancer Res. 70(14):5880-5890, 2010; Lin et al., Thorax 69(2):152-160, 2014; Cisowski et al., Am. J. Pathol. 179(1):513-523, 2011; Rana et al., Arterioscler. Thromb. Vasc. Biol. 38(6):1368-1380, 2018). PZ-128 (P1 pal-7), has advanced through phase 1 clinical studies in subjects with coronary artery disease, where it was shown to be a safe and effective first-in-class pepducin inhibitor of PAR1 that suppresses platelet activation without effects on hemostasis (Gurbel et al., Arterioscler. Thromb. Vasc. Biol. 36(1):189-197, 2016). More recently, a placebo-controlled phase 2 study with PZ-128 was successfully completed in 2020 in the US in patients who underwent cardiac catheterization and stenting or subsequent coronary artery bypass surgery (CABG) to treat coronary artery disease (Kuliopulos et al., Arterioscler. Thromb. Vasc. Biol 40(12):2990-3003, 2020).

Despite existing therapies, the prevalence of heart failure (HF) is projected to increase by nearly 50% from 2012 to 2030, affecting >8 million adults (Virani et al, Circulation 143(8):e254-e743, 2021). Life-time risks for developing HF range from 20% to 45% in patients over 45 years old. Among 110,621 patients hospitalized with HF between 2005 to 2010, LV ejection fraction (EF) was reduced (<40%) in half, intermediate EF (40-50%) in 14%, and preserved EF (≥50%) in 36% (Virani et al, Circulation 143(8):e254-e743, 2021). Although generally not disease-modifying, current treatment of heart failure with reduced ejection fraction (HFrEF) typically consists of ACE inhibitors, angiotensin receptor neprilysin inhibitor (ARNI), β-blockers, coronary revascularization, devices, and most recently empagliflozin, a SGLT2 inhibitor. By comparison, patients with HF with preserved ejection fraction (HFpEF), have less adequate treatment options to prevent eventual progression of their disease.

Cardiac fibrosis is central to the pathogenesis of heart failure, in particular for those with preserved ejection fraction. Irrespective of the underlying profibrotic condition, maladaptive cardiac fibrosis is defined by the transformation of resident fibroblasts to matrix-secreting myofibroblasts. Numerous profibrotic factors have been identified (e.g., TGFβ, IL11, AngII), which activate gene expression programs for myofibroblast activation. A number of existing therapies indirectly target upstream factors such as diabetes and hypertension, but a clinically effective anti-fibrotic therapy remains elusive. Notably, therapeutic inhibition of TGFβ, the master-regulator of fibrosis, has proven toxic and ineffective in clinical trials to date, and new approaches are needed.

### SUMMARY

In some aspects, the invention provides methods of providing direct cardioprotection to a subject, the method comprising administering PZ-128 to the subject. The invention also provides methods for preventing, reducing, or treating myocardial stress in a subject, the method comprising administering PZ-128 to the subject.

In some embodiments, the PZ-128 is administered parenterally (e.g., intravenously).

In some embodiments, the myocardial stress is assessed by measurement of B-type (Brain) Natriuretic Peptide (BNP) or an N-terminal fragment thereof in a sample from the subject, and a reduction in BNP or an N-terminal fragment thereof of indicates that the treatment has resulted in prevention, reduction, or treatment of myocardial stress in the subject.

In some embodiments, the subject has or is at risk of developing heart failure (HF), which may optionally be, e.g., HF with preserved ejection fraction (HFpEF), HF with reduced ejection fraction (HFrEF), or intermediate HF.

In some embodiments, the subject has recently or is currently experiencing acute coronary syndrome (ACS).

In some embodiments, the subject has recently or is currently experiencing ST-segment-elevation myocardial infarction (STEMI).

In some embodiments, the subject has recently or is currently experiencing Non-ST-segment-elevation myocardial infarction (NSTEMI).

In some embodiments, the subject has recently or is currently experiencing unstable angina.

In some embodiments, the treatment is carried out in an acute care setting within 3 days of symptom onset.

In some embodiments, the treatment is carried out for five or fewer days, for example, one to three days.

In some embodiments, the subject is administered PZ-128 once daily for three days.

In some embodiments, the subject is administered PZ-128 for 1-4 weeks or more, for example, for up to 1, 2, or 3 months.

In some embodiments, the PZ-128 is administered once daily, every 2 days, every 3 days, twice weekly, once weekly, biweekly, or monthly.

In some embodiments, the method improves ejection fraction (EF) in the subject.

In some embodiments, the method improves fractional shortening by echocardiogram in a subject.

In some embodiments, the method improves left ventricular function in the subject.

In some embodiments, the method improves coronary microvascular blood flow.

In some embodiments, the method increases reperfusion area in the heart of the subject.

In some embodiments, the method decreases myonecrosis in the heart of the subject.

In some embodiments, the method decreases infarct size in the heart of the subject.

In some embodiments, the subject has or is at risk of developing cardiac fibrosis, and the treatment prevents, reduces, or treats the cardiac fibrosis.

In some embodiments, the subject is characterized by elevated levels of cardiac troponins.

In some embodiments, the parenteral administration is intravenous administration.

In some embodiments, the subject is administered 0.1 mg/kg-1.0 mg/kg PZ-128 by 1-4 hours of intravenous infusion.

In some embodiments, the subject is administered about 0.3 or about 0.5 mg/kg PZ-128 by intravenous infusion for about 2 hours.

In some embodiments, the method further comprises thrombolytic therapy.

In some embodiments, the subject is receiving cardiac catheterization.

In some embodiments, the method further comprises percutaneous coronary intervention (PCI).

In some embodiments, the administration is initiated within 1 hour before percutaneous coronary intervention (PCI) and optionally is carried out in combination with oral antiplatelet therapy.

In some embodiments, the administration is carried out in a subject who is not treated by PCI.

In some embodiments, the subject is treated by coronary artery bypass graft (CABG) after PZ-128 treatment and is not treated by PCI.

In some embodiments, the methods further comprise administration of one or more drug selected from the following categories: anti-hypertensives, angiotensin receptor-neprilysin inhibitors (e.g., sacubitril/valsartan; Entresto), sodium-glucose co-transporter-2 (SGLT2) inhibitors (e.g., canagliflozin, dapagliflozin, and empagliflozin); angiotensin receptor blockers (e.g., losartan, candesartan, and telmesartan), renin-angiotensin antagonists (ARBs, ACE inhibitors (e.g., enalapril, captopril and ramipril)), beta blockers (e.g., atenolol, metoprolol, nadolol, pindolol, carvedilol, and labetelol), mineralocorticoid receptor antagonists (e.g., spironolactone, eplerenone, canrenone, finerenone, and mexrenone), diuretics (e.g., hydroclorathiazide, thiazide, indapamide, and chlorathalidone), hydralazine/nitrates (e.g., BiDil; isosorbide dinitrate and hydralazine hcl), and calcium channel blockers (e.g., amlodipine, nifedipine, nicardipine and verapamil)

In some embodiments, the method further comprises treatment of the subject with a ventricular assist devise, such as IMPELLA.

In some embodiments, the subject is at high risk for bleeding.

In some embodiments, the subject is 75 years old or older.

In some embodiments, the subject is not treated with aspirin.

In further aspects, the invention further provides methods of providing direct cardioprotection to a subject, the method comprising administering palmitate-KKSRALF-NH₂ or a pharmaceutically acceptable salt thereof (e.g., the acetate salt) to the subject. The invention also provides methods for preventing, reducing, or treating myocardial stress in a subject, the method comprising administering palmitate-KKSRALF-NH₂ or a pharmaceutically acceptable salt thereof (e.g., the acetate salt) to the subject.

In some embodiments, the palmitate-KKSRALF-NH₂ or a pharmaceutically acceptable salt thereof (e.g., the acetate salt) is administered parenterally (e.g., intravenously).

In some embodiments, the myocardial stress is assessed by measurement of B-type (Brain) Natriuretic Peptide (BNP) or an N-terminal fragment thereof in a sample from the subject, and a reduction in BNP or an N-terminal fragment thereof of indicates that the treatment has resulted in prevention, reduction, or treatment of myocardial stress in the subject.

In some embodiments, the subject has or is at risk of developing heart failure (HF), which may optionally be, e.g., HF with preserved ejection fraction (HFpEF), HF with reduced ejection fraction (HFrEF), or intermediate HF.

In some embodiments, the subject has recently or is currently experiencing acute coronary syndrome (ACS).

In some embodiments, the subject has recently or is currently experiencing ST-segment-elevation myocardial infarction (STEMI).

In some embodiments, the subject has recently or is currently experiencing Non-ST-segment-elevation myocardial infarction (NSTEMI).

In some embodiments, the subject has recently or is currently experiencing unstable angina.

In some embodiments, the treatment is carried out in an acute care setting within 3 days of symptom onset.

In some embodiments, the treatment is carried out for five or fewer days, for example, one to three days.

In some embodiments, the subject is administered palmitate-KKSRALF-NH₂ or a pharmaceutically acceptable salt thereof (e.g., the acetate salt) once daily for three days.

In some embodiments, the subject is administered palmitate-KKSRALF-NH₂ or a pharmaceutically acceptable salt thereof (e.g., the acetate salt) for 1-4 weeks or more, for example, for up to 1, 2, or 3 months.

In some embodiments, the palmitate-KKSRALF-NH₂ or a pharmaceutically acceptable salt thereof (e.g., the acetate salt) is administered once daily, every 2 days, every 3 days, twice weekly, once weekly, biweekly, or monthly.

In some embodiments, the method improves ejection fraction (EF) in the subject.

In some embodiments, the method improves fractional shortening by echocardiogram in a subject.

In some embodiments, the method improves left ventricular function in the subject.

In some embodiments, the method improves coronary microvascular blood flow.

In some embodiments, the method increases reperfusion area in the heart of the subject.

In some embodiments, the method decreases myonecrosis in the heart of the subject.

In some embodiments, the method decreases infarct size in the heart of the subject.

In some embodiments, the subject has or is at risk of developing cardiac fibrosis, and the treatment prevents, reduces, or treats the cardiac fibrosis.

In some embodiments, the subject is characterized by elevated levels of cardiac troponins.

In some embodiments, the parenteral administration is intravenous administration.

In some embodiments, the subject is administered 0.1 mg/kg-1.0 mg/kg palmitate-KKSRALF-NH₂ or a pharmaceutically acceptable salt thereof (e.g., the acetate salt) by 1-4 hours of intravenous infusion.

In some embodiments, the subject is administered about 0.3 or about 0.5 mg/kg palmitate-KKSRALF-NH₂ or a pharmaceutically acceptable salt thereof (e.g., the acetate salt) by intravenous infusion for about 2 hours.

In some embodiments, the method further comprises thrombolytic therapy.

In some embodiments, the subject is receiving cardiac catheterization.

In some embodiments, the method further comprises percutaneous coronary intervention (PCI).

In some embodiments, the administration is initiated within 1 hour before percutaneous coronary intervention (PCI) and optionally is carried out in combination with oral antiplatelet therapy.

In some embodiments, the administration is carried out in a subject who is not treated by PCI.

In some embodiments, the subject is treated by coronary artery bypass graft (CABG) after palmitate-KKSRALF-NH₂ or a pharmaceutically acceptable salt thereof (e.g., the acetate salt) treatment and is not treated by PCI.

In some embodiments, the methods further comprise administration of one or more drug selected from the following categories: anti-hypertensives, angiotensin receptor-neprilysin inhibitors (e.g., sacubitril/valsartan; Entresto), sodium-glucose co-transporter-2 (SGLT2) inhibitors (e.g., canagliflozin, dapagliflozin, and empagliflozin); angiotensin receptor blockers (e.g., losartan, candesartan, and telmesartan), renin-angiotensin antagonists (ARBs, ACE inhibitors (e.g., enalapril, captopril and ramipril)), beta blockers (e.g., atenolol, metoprolol, nadolol, pindolol, carvedilol, and labetelol), mineralocorticoid receptor antagonists (e.g., spironolactone, eplerenone, canrenone, finerenone, and mexrenone), diuretics (e.g., hydroclorathiazide, thiazide, indapamide, and chlorathalidone), hydralazine/nitrates (e.g., BiDil; isosorbide dinitrate and hydralazine hcl), and calcium channel blockers (e.g., amlodipine, nifedipine, nicardipine and verapamil) In some embodiments, the method further comprises treatment of the subject with a ventricular assist devise, such as IMPELLA.

In some embodiments, the subject is at high risk for bleeding.

In some embodiments, the subject is 75 years old or older.

In some embodiments, the subject is not treated with aspirin.

In some embodiments, the compound administered is palmitate-KKSRALF-NH₂ (PK-128).

In some embodiments, the compound administered is a pharmaceutically acceptable salt of palmitate-KKSRALF-NH₂ (PK-128).

In some embodiments, the compound administered is the acetate salt of palmitate-KKSRALF-NH₂ (PK-128).

In some embodiments, the palmitate KKSRALF-NH₂, or pharmaceutically acceptable salt thereof, is in the form of a micelle.

### BRIEF DESCRIPTION OF THE DRAWINGS

FIGS. 1A-E show protection against cardiac myocardial injury by PZ-128 in mice. FIG. 1A shows digital scans of sections. FIG. 1B shows infarct area (=I), FIG. 1C shows Area at Risk (=AAR), FIG. 1D shows Total Ischemic area, and FIG. 1E shows reperfusion area (=R). Images were quantified using ImageJ and adjusted by weight of each cardiac slice. Data are shown as mean±SD and analyzed by two-tailed Student's t-test: **p<0.01, ***p<0.001.

FIG. 2 shows echocardiography of mouse hearts after 3 days reperfusion following 30 minutes myocardial ischemia treated with vehicle or 10 mg/kg PZ-128.

FIG. 3 shows NT-proBNP plasma levels 24 hours after PZ-128 or placebo infusion in patients with the diagnosis of heart failure (HFpEF or HFrEF) undergoing cardiac catheterization or percutaneous coronary intervention (PCI) in the TRIP-PCI study. Data was analyzed by 2-sided paired T-tests. Normal levels of NT-proBNP ≤125 pg/mL.

FIGS. 4A-4H show that PZ-128 protects against cardiac fibrosis and ventricular hypertrophy and improves LV function in a pressure-overload (TAC) model of HFrEF in mice. Male C57BL/6N mice were treated daily for 7 weeks with vehicle or 10 mg/kg PZ-128 sc following 25G transaortic constriction (TAC) or sham surgery (Richards et al., Scientific Reports 9(1):5844, 2019) (n=5-7). (FIG. 4A) Photomicrographs of interstitial fibrosis in cardiac sections stained with sirius red from Sham and TAC animals treated with Veh or PZ-128. (FIG. 4B) Interstitial collagen area from animals in A was quantified along with (FIG. 4C) Col1a1 expression by Q-PCR. (FIGS. 4D-4E) LV fractional shortening and ejection fraction were quantified by ECHO. (FIG. 4F) The outline of cardiomyocytes was identified by wheat germ agglutinin (WGA-green) staining with (FIG. 4G) mean myocyte area digitally quantified. (FIG. 4H) Mean weight (mg) of LV normalized to tibia length (mm).

FIGS. 5A-5C show that PZ-128 suppresses leukocyte infiltration into cardiac muscle in the TAC model in mice and inhibits human PBMC collagen invasion. (FIG. 5A) Photomicrographs of CD11 b+monocytes/macrophages and CD4+ T cells in Sham vs TAC in mice treated with vehicle or PZ-128 as in FIGS. 4A-4H, with CD11 b staining area quantified in FIG. 5B and T cells quantified in FIG. 5C.

FIG. 6 shows that PZ-128 suppresses enhanced fibrotic and inflammatory gene expression in the 7-week TAC model of HF (n=5-7) as assessed by quantitative Q-PCR relative to GAPDH mRNA.

### DETAILED DESCRIPTION

The invention provides methods of providing direct cardioprotection to the heart of a subject. In some embodiments, the subject has recently or is currently experiencing acute coronary syndrome (ACS), ST-segment-elevation myocardial infarction (STEMI), Non-ST-segment-elevation myocardial infarction (NSTEMI), and/or unstable angina. In some embodiments, the treatment of these subjects is carried out in an acute care setting, as explained further below. In some embodiments, the direct cardioprotection is provided to the heart without the need for involvement of platelets.

The invention additionally provides methods of preventing, inhibiting, reducing, delaying, or treating myocardial stress or the effects thereof in a subject. In some embodiments, the myocardial stress is associated with increased levels of B-type, or brain, natriuretic peptide (BNP; e.g., levels over 100 pg/mL in patients not being treated with sacubitril/valsartan (Entresto)) or an N-terminal fragment thereof (e.g., NT-proBNP; e.g., levels over 900 pg/mL) in a sample from a subject (e.g., a blood sample).

In some embodiments, the myocardial stress is a mechanical stress. In some embodiments, the subject has or is at risk of developing heart failure (HF). The methods of the invention can thus be used to prevent, inhibit, reduce, delay, or treat heart failure (HF) in a subject. In some embodiments, the heart failure is heart failure with preserved ejection fraction (HFpEF). In some embodiments, the heart failure is heart failure with reduced ejection fraction (HFrEF). In some embodiments, the heart failure is characterized by an intermediate ejection fraction. In some embodiments, the subjects with HFpEP have left ventricular ejection fraction (LVEF) of ≥50 percent. In some embodiments, the subjects with HFrEF have a LVEF of ≤40 percent. In some embodiments, the subjects with an intermediate ejection fraction have a LVEF falling in the intermediate range (>40 to <50 percent). Techniques used to measure LVEF include, e.g., echocardiography, magnetic resonance imaging (MRI), computed tomography (CT), radionuclide angiography, gated myocardial perfusion single-photon emission computed tomography (SPECT), and gated myocardial perfusion positron emission tomography (PET), as are known in the art. LVEF can be calculated by subtracting the end-systolic LV volume from the end-diastolic LV volume and then dividing by the end-diastolic LV volume, as is known in the art. In some embodiments, these methods are carried out in an acute care setting. In some embodiments, the methods are carried out chronically.

The methods described above and further herein involve the administration of a molecule designated herein as PZ-128, which is a cell-penetrating lipopeptide derived from the juxtamembrane region of the 3 loop and N-terminus of transmembrane domain 6 (TM6) of PAR1 (see, e.g., U.S. Pat. No. 9,878,054). PZ-128 comprises the structure of palmitate-KKSRALF-NH₂, which is set forth in an exemplary manner in further detail below. When use of PZ-128 is mentioned herein it is to be understood that the PZ-128 may optionally be formulated, e.g., as described herein, e.g., as a pharmaceutically acceptable salt thereof, e.g., as described herein (e.g., an acetate salt thereof), which may be in the form of micelles. Non-limiting details of the methods of the invention, as well as compositions used in the invention, are described further as follows.

### Terms

Unless defined otherwise, all technical and scientific terms used herein have the same meaning as is commonly understood by one of skill in the art. The following definitions are provided to help interpret the disclosure and claims of this application. In the event a definition in this section is not consistent with definitions elsewhere, the definition set forth in this section will control.

As used herein, “pepducin compounds” are cell-penetrating peptides that act as intracellular agonists or antagonist of signal transference from receptors to G proteins. Pepducin compounds utilize lipidated fragments of intracellular G protein-coupled receptor loops to modulate GPCR action in targeted cell-signaling pathways. A pepducin compound comprises a short polypeptide derived from a GPCR intracellular loop tethered to a hydrophobic moiety. This structure allows pepducin compounds to anchor in the cell membrane lipid bilayer and target the GPCR/G protein interface via a unique intracellular allosteric mechanism. Examples of pepducin compounds are described in U.S. Patent Publication US 2007/0179090, the contents of which are hereby incorporated herein by reference in its entirety. Palmitate-KKSRALF-NH₂ (PZ-128) may be depicted as a molecule with the following structure:

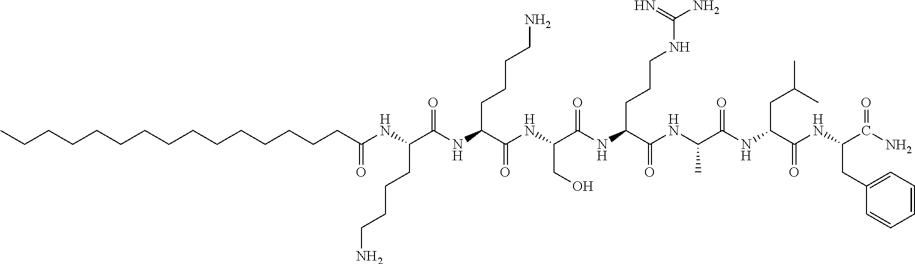 [SMILES: CCCCCCCCCCCCCCCC(=O)N[C@@H](CCCCN)C(=O)N[C@@H](CCCCN)C(=O)N[C@@H](CO)C(=O)N[C@@H](CCCNC(=N)N)C(=O)N[C@@H](C)C(=O)N[C@H](CC(C)C)C(=O)N[C@@H](Cc1ccccc1)C(N)=O]

or, e.g., a corresponding flat version thereof, in the absence of stereochemical indications. Palmitate-KKSRALF-NH₂ (PZ-128) may optionally be formulated in the form of a pharmaceutically acceptable salt, such as an acetate salt.

The terms “palmitate-KKSRALF-NH₂ acetic acid salts” with regard to a molecular weight refer to the molecular weight of palmitate-KKSRALF-NH₂ plus acetic acid counterions.

“Subject” means any animal, such as a human patient, livestock, or domestic pet.

As used herein, the terms “prevent” and “preventing” include the prevention of recurrence, spread, or onset. It is not intended that the present disclosure be limited to complete prevention. In some embodiments, the onset is delayed, or the severity of the disease is reduced.

As used herein, the terms “treat” and “treating” are not limited to the case where the subject (e.g., human patient) is cured and the disease is eradicated. Rather, embodiments of the present disclosure also contemplate treatment that merely reduces one or more symptoms and/or delays disease progression.

As used herein, the term “about” or “approximately” when used in conjunction with a number refers to any number within 5, 10, or 15% of the referenced number.

As used herein the terms “administration,” “administering,” or the like, when used in the context of providing a pharmaceutical composition to a subject, generally refers to providing to the subject one or more pharmaceutical compositions comprising the agent, e.g., micelle particles of PZ-128, in combination with an appropriate delivery vehicle by any means such that the administered compound achieves one or more of the intended biological effects for which the compound was administered. By way of non-limiting example, a composition may be administered by parenteral, subcutaneous, intravenous, intracoronary, rectal, intramuscular, intra-peritoneal, transdermal, or buccal routes of delivery.

The term “polypeptide” refers to any chain or chains of two or more amino acids and does not refer to a specific length of the product. A “peptide” or “polypeptide” as used herein, may be derived from a natural biological source, synthesized, or produced by recombinant technology. It may be generated in any manner, including by chemical synthesis. In accordance with this definition, a “polypeptide” may be of a size of about 3 or more, about 5 or more, about 10 or more, about 20 or more, about 25 or more, about 50 or more, about 75 or more, about 100 or more, about 200 or more, about 500 or more, about 1,000 or more, or about 2,000 or more amino acids. One or more of the amino acids may be modified, for example, by the addition of a chemical entity such as a carbohydrate group, a phosphate group, a farnesyl group, an isofarmesyl group, a fatty acid group, an acyl group (e.g., acetyl group), a linker for conjugation, functionalization, or other known protecting/blocking groups. A “polypeptide,” as used herein, may be fragments, derivatives, analogs, or variants of the foregoing polypeptides, and any combination thereof. Fragments of polypeptides, as that term or phrase is used herein, include proteolytic fragments, as well as deletion fragments. Variants of polypeptides include fragments and polypeptides with altered amino acid sequences due to amino acid substitutions, deletions, or insertions. Variants may occur naturally or be non-naturally occurring. Examples include fusion proteins, polypeptides having one or more residues chemically derivatized by reaction of a functional side group, and peptides that contain one or more naturally occurring amino acid derivatives of the twenty standard amino acids. These modifications may also include the incorporation of D-amino acids, or other non-encoded amino-acids. None of the modifications should substantially interfere with the desired biological activity of the peptide.

### Micelles

In certain embodiments, the disclosure includes micelle particles of polypeptide and lipophilic moiety conjugates (e.g., PZ-128) in substantially pure form and their preparation. Stored polypeptide and lipophilic moiety conjugates have a tendency to degrade over time. Certain micelle compositions disclosed herein have superior stabilization properties due to the manner in which they are prepared.

Aqueous pharmaceutical compositions comprising PZ-128 (e.g., micelles of palmitate-KKSRALF-NH₂ acetic acid salts) can take several different forms, e.g., aggregates and particle forms, and sizes due to the presence of surrounding water, acidic condition, and added excipients. Aggregate and particle forms alter stability. For the purpose of administering the pharmaceutical composition to a subject, it is important that the particle sizes and makeup are consistent and substantially similar so that the pharmacokinetic profile after administration is not altered when exposed to components in blood serum.

Methods for making PZ-128, micelles, and formulations including pharmaceutically acceptable salts thereof, as well as related formulations, which can be used in the present invention, are described, e.g., in U.S. Pat. No. 9,878,054.

### Pharmaceutical Compositions and Methods

In certain embodiments, the disclosure relates to pharmaceutical composition comprising micelle particles disclosed herein and a pharmaceutically acceptable excipient. Micelles comprising polypeptide and lipophilic moiety conjugate salts, e.g., palmitate-KKSRALF-NH₂ micelle particles made up of pharmaceutically acceptable salts are also useful in the method of the disclosure and in pharmaceutical compositions of the disclosure. The pharmaceutical compositions of the present disclosure can be administered to subjects either orally, rectally, parenterally (intravenously, intramuscularly, or subcutaneously), intracistemally, intravaginally, intraperitoneally, intravesically, locally (powders, ointments, or drops), or as a buccal or nasal spray. In another option, intracoronary administration may be used, by which injection is done directly from a catheter into coronary artery circulation. This can be done concurrently with coronary device use or device insertion, such as a coronary assist device (e.g., Impella or PCI).

In certain embodiments, the disclosure relates to micelles comprising polypeptide and lipophilic moiety conjugate salts, e.g., micelle particles comprising palmitate-KKSRALF-NH₂ salts wherein the counterion is selected from adipic acid, camphoric acid, carbonic acid, cinnamon acid, citric acid, fumaric acid, galactaric acid, gentisic acid, glucaric acid, glucoheptonic acid, D-gluconic acid, D-glucuronic acid, gluataric acid, alpha-oxo-glutaric acid, lactobionic acid, maleic acid, L-malic acid, malonic acid, pamoic acid, pyruvic acid, salicylic acid, sebacic acid, succinic acid, tartaric acid, or combinations thereof. In certain embodiments, the disclosure relates to palmitate-KKSRALF-NH₂ salts wherein the counterion is ascorbic acid or acetic acid. In certain embodiments, the salt may be in a composition optionally comprising sodium ion, ammonium, imidazole, or combinations thereof.

In some embodiments, the pharmaceutical composition is an aqueous solution comprising a saccharide or polysaccharide (e.g., dextrose) at about or less than 5% by weight

In some embodiments, palmitate-KKSRALF-NH₂ is administered in a composition comprising dextrose as a pharmaceutically acceptable excipient.

In some embodiments, the invention employs a pharmaceutical composition comprising micelles of palmitate-KKSRALF-NH₂ acid salts and an aqueous solution comprising dextrose at about 5% by weight.

In some embodiments, palmitate-KKSRALF-NH₂ acid salts are in the form of acetic acid salts.

In some embodiments, the micelle averages one, two, or three acetic acid counter anions per palmitate-KKSRALF-NH₂ cation.

In some embodiments, palmitate-KKSRALF-NH₂ (48 mg lyophilized powder/20 cc glass vial solubilized with 10 mL sterile water) in the form of micelles diluted into 5% dextrose/95% water (D5W), pH ˜6, as an acetate salt, in 250 cc of sterile D5W for an adjusted dose of 0.3 to 0.5 mg PZ-128/kg (body weight) may be used.

In some embodiments, a composition comprising palmitate-KKSRALF-NH₂, e.g., as described herein is administered daily, every other day, every 3 days, twice weekly, or once weekly by, e.g., intravenous infusion, which optionally is carried out over a period of about 2 hours (e.g., 1-4, 1.5-3.5, or 2-3 hours). In some embodiments, such a composition is by intravenous infusion over a period of about 2 hours. In some embodiments, such a composition is administered by intravenous (iv) infusion over a period of about 2 hours for up to seven iv infusions over a 1 month period. In some embodiments, such a composition is administered (e.g., by intravenous infusion for about 2 hours) every 2-3 days during hospitalization and once weekly post discharge, not to exceed 7 doses per month.

In certain embodiments, the disclosure relates to pharmaceutical compositions comprising palmitate-KKSRALF-NH₂ salts in combination with mannitol, glucuronic acid, or combinations thereof. Micelle particles suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents solvents or vehicles include water, ethanol, polyols (propylene glycol, polyethylene glycol, glycerol, and the like), suitable mixtures thereof, vegetable (such as olive oil, sesame oil and viscoleo) and injectable organic esters such as ethyl oleate. Proper fluidity can be maintained, for example, by the use of a coating such as lecithin, by the maintenance of the required particle size in the case of dispersions and by the surfactants. These compositions may also contain adjuvants such as preserving, emulsifying, and dispensing agents. Prevention of the action of microorganisms be controlled by addition of any of various antibacterial and antifungal agents, example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.

Solid dosage forms for oral administration include capsules, tablets, pills, powders and granules. In such solid dosage forms, the micelle particles are admixed with at least one inert customary excipient (or carrier) such as sodium citrate or dicalcium phosphate or: (a) fillers or extenders, as for example, starches, lactose, sucrose, glucose, mannitol and silicic acid, (b) binders, as for example, carboxymethylcellulose, alginates, gelatin, polyvinylpyrrolidone, sucrose, and acacia, (c) humectants, as for example, glycerol (d) disintegrating agents, as for example, agar-agar, calcium carbonate, potato or tapioca starch, alginic acid, certain complex silicates, and sodium carbonate, (e) solution retarders, as for example paraffin, (f) absorption accelerators, as for example, quaternary ammonium compounds, (g) wetting agents, as for example cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar and as high molecular weight polyethylene glycols, and the like.

Solid dosage forms such as tablets, dragees, capsules, pills, and granules can be prepared with coatings and shells, such as enteric coatings and others well known in the art. They may contain opacifying agents and can also be of such composition that they release palmitate-KKSRALF-NH₂ or salts in a certain part of the intestinal tract in a delayed manner. Examples of embedding compositions which can be used are polymeric substances and waxes. The micelle particles can also be used in micro-encapsulated form, if appropriate, with one or more of the above-mentioned excipients. Controlled slow-release formulations are also preferred, including osmotic pumps and layered delivery systems.

Liquid dosage forms for oral administration include pharmaceutically acceptable emulsions, solutions, suspensions, syrups, and elixirs. In addition to the micelles comprising polypeptide and lipophilic moiety conjugate salts, e.g., palmitate-KKSRALF-NH₂ salts, the liquid dosage forms may contain inert diluents commonly used in the art, such as water or other solvents, solubilizing agents and emulsifiers, for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, viscoleo, castor oil and sesame oil, glycerol, tetrahydrofurfuryl alcohol, poly ethylene glycols and fatty acid esters of sorbitan or mixtures of these substances, and the like.

Besides such inert diluents, the composition can also include adjuvants, such as wetting agents, emulsifying and suspending agents, sweetening, flavoring, and perfuming agents. Suspensions, in addition to micelles comprising polypeptide and lipophilic moiety conjugate salts, e.g., palmitate-KKSRALF-NH₂ salts, may contain suspending agents, as for example, ethoxylated iso-stearyl alcohols, polyoxyethylene sorbitol and sorbitan esters, microcrystalline cellulose, aluminum metahydroxide, bentonite agar-agar and tragacanth, or mixtures of these substances, and the like.

Pharmaceutical compositions disclosed herein can be formulated in the form of pharmaceutically acceptable salts, as generally described below. Some preferred, but non-limiting examples of suitable pharmaceutically acceptable organic and/or inorganic acids are acetic acid, hydrochloric acid, hydrobromic acid, sulfuric acid, nitric acid, and citric acid, adipic acid, camphoric acid, carbonic acid, cinnamon acid, citric acid, fumaric acid, galactaric acid, gentisic acid, glucaric acid, glucoheptonic acid, D-gluconic acid, D-glucuronic acid, gluataric acid, alpha-oxo-glutaric acid, lactobionic acid, maleic acid, L-malic acid, malonic acid, pamoic acid, pyruvic acid, salicylic acid, sebacic acid, succinic acid, tartaric acid, or combinations thereof.

Pharmaceutically acceptable salts of polypeptide and lipophilic moiety conjugates, e.g., palmitate-KKSRALF-NH₂, include the acid addition and base salts thereof. Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases can also be formed, for example, hemisulphate and hemicalcium salts. For a review on suitable salts, see Handbook of Pharmaceutical Salts: Properties, Selection, and Use by Stahl and Wermuth (Wiley-VCH, 2002), incorporated herein by reference.

Polypeptide and lipophilic moiety conjugate salts, e.g., palmitate-KKSRALF-NH₂ salts described herein, can be administered in the form of prodrugs. A prodrug can include a covalently bonded carrier which releases the active parent drug when administered to a mammalian subject. Prodrugs can be prepared by modifying functional groups present in palmitate-KKSRALF-NH₂ in such a way that the modifications are cleaved, either in routine manipulation or in vivo, to the parent compounds. Prodrugs include, for example, wherein a hydroxyl group is bonded to any group that, when administered to a mammalian subject, cleaves to form a free hydroxyl group. Examples of prodrugs include, but are not limited to, acetate, formate and benzoate derivatives of alcohol functional groups in palmitate-KKSRALF-NH₂. Examples of structuring a compound as prodrugs can be found in the book of Testa and Caner, Hydrolysis in Drug and Prodrug Metabolism, Wiley (2006) hereby incorporated by reference. Typical prodrugs form the active metabolite by transformation of the prodrug by hydrolytic enzymes, the hydrolysis of amides, lactams, peptides, carboxylic acid esters, epoxides or the cleavage of esters of inorganic acids.

Pharmaceutical compositions typically comprise an effective amount of micelles particles of polypeptide and lipophilic moiety conjugate salts, e.g., palmitate-KKSRALF-NH₂ salts and a suitable pharmaceutical acceptable carrier. The preparations can be prepared in a manner known per se, which usually involves mixing micelles with the one or more pharmaceutically acceptable carriers, and, if desired, in combination with other pharmaceutical active compounds, when necessary, under aseptic conditions. Reference is made to U.S. Pat. Nos. 6,372,778, 6,369,086, 6,369,087, and 6,372,733, and the further references mentioned above, as well as to the standard handbooks, such as the latest edition of Remington's Pharmaceutical Sciences. Ester prodrugs are readily degraded in the body to release the corresponding alcohol. See e.g., Imai, Drug Metab Pharmacokinet. (2006) 21(3):173-185, entitled “Human carboxylesterase isozymes: catalytic properties and rational drug design.

In certain embodiments, for pharmaceutical use, micelle particles of palmitate-KKSRALF-NH₂ salts can be formulated as a pharmaceutical preparation comprising palmitate-KKSRALF-NH₂ salts and at least one pharmaceutically acceptable carrier, diluent, or excipient and/or adjuvant, and optionally one or more further pharmaceutically active compounds. The pharmaceutical preparations of the disclosure are preferably in a unit dosage form, and can be suitably packaged, for example, in a box, blister, vial, bottle, sachet, ampoule or in any other suitable single-dose or multi-dose holder or container (which can be properly labeled); optionally with one or more leaflets containing product information and/or instructions for use. Generally, such unit dosages will contain between 1 and 1000 mg, and usually between 5 and 500 mg, micelle particles of palmitate-KKSRALF-NH₂ salts of the disclosure e.g., about 10, 25, 50, 100, 200, 300, or 400 mg per unit dosage.

The micelle particles will generally be administered in an “effective amount,” by which it is meant any amount of palmitate-KKSRALF-NH₂ salts disclosed herein that, upon suitable administration, is sufficient to achieve the desired therapeutic or prophylactic effect in the subject to which it is administered. Usually, depending on the condition to be prevented or treated and the route of administration, such an effective amount will usually be between 0.01 to 1000 mg per kilogram body weight of the subject per day, more often between 0.1 and 500 mg, such as between 1 and 250 mg, for example about 5, 10, 20, 50, 100, 150, 200, or 250 mg, per kilogram body weight of the subject per day, which can be administered as a single daily dose, divided over one or more daily doses. The amount(s) to be administered, the route of administration and the further treatment regimen can be determined by the treating clinician, depending on factors such as the age, gender and general condition of the subject and the nature and severity of the disease/symptoms to be treated. Reference is made to U.S. Pat. Nos. 6,372,778, 6,369,086, 6,369,087, and 6,372,733 and the further references mentioned above, as well as to the standard handbooks, such as the latest edition of Remington's Pharmaceutical Sciences.

As explained above, in some embodiments, treatment is in an acute setting. In some embodiments, treatment is carried out within 1, 2, 3, 4, or 5 days (e.g., 3 days) of symptom onset. In some embodiments, the treatment is carried out for 5 or fewer days, e.g., for 4, 3, 2, or 1 day. In some embodiments, the treatment according to these methods is carried out once on each of the treatment days. In some embodiments, the treatment is carried out in a relatively long term, chronic context. In some embodiments, the treatment is carried out for 1-4 weeks (e.g., 1, 2, 3, or 4 weeks) or more, for example, for up to 1, 2, 3, 4, 5, or 6 months. In some embodiments, the treatment according to these methods is carried out once daily, every 2 days, every 3 days, twice weekly, once weekly, biweekly, or monthly. The dosage regimen used can be selected by those of skill in the art depending upon, e.g., the factors noted in the prior paragraph.

PZ-128, which may be formulated as a pharmaceutically acceptable salt thereof, e.g., as described herein, can be administered as a sole therapeutic agent or, alternatively, can be administered with another therapeutic agent or in the context of another therapeutic treatment.

In some embodiments, treatment according to the methods of the invention, as described herein, is carried out in combination with one or more other treatments. Thus, in some embodiments, PZ-128 is administered in combination with one or more drug selected from the following categories: anti-hypertensives, angiotensin receptor-neprilysin inhibitors, sodium-glucose co-transporter-2 (SGLT2) inhibitors, angiotensin receptor blockers, renin-angiotensin antagonists, beta blockers, mineralocorticoid receptor antagonists, diuretics, hydralazine/nitrates, and calcium channel blockers.

In more detail, the combination(s) may be with: angiotensin receptor-neprilysin inhibitors (e.g., sacubitril/valsartan; Entresto), sodium-glucose co-transporter-2 (SGLT2) inhibitors (e.g., canagliflozin, dapagliflozin, and empagliflozin); angiotensin receptor blockers (e.g., losartan, candesartan, and telmesartan), renin-angiotensin antagonists (ARBs, ACE inhibitors (e.g., enalapril, captopril and ramipril)), beta blockers (e.g., atenolol, metoprolol, nadolol, pindolol, carvedilol, and labetelol), mineralocorticoid receptor antagonists (e.g., spironolactone, eplerenone, canrenone, finerenone, and mexrenone), diuretics (e.g., hydroclorathiazide, thiazide, indapamide, and chlorathalidone), hydralazine/nitrates (e.g., BiDil; isosorbide dinitrate and hydralazine hcl), and/or calcium channel blockers (e.g., amlodipine, nifedipine, nicardipine and verapamil).

In some embodiments, the methods are carried out in combination with left ventricular (LV) decompression. In some embodiments, LV decompression is carried out using percutaneous approach such as, for example, an intra-aortic balloon pump (IABP), a trans-valvular pump (TVP), a trans-aortic LV assist device (e.g., Impella®, Abiomed, Danvers, MA), trans-aortic drainage catheters, left atrial decompression (e.g., TandemHeart), pulmonary artery drainage cannulas, trans-septal left atrial pulmonary artery and trans-aortic LV venting. In some embodiments, LV decompression is carried out using surgical means, e.g., atrial septostomy or direct surgical LV, LA, and pulmonary artery venting.

The following Examples are intended to illustrate certain embodiments of the invention and are not intended to be limiting of the scope thereof.

### Example 1

TRIP-PCI was a multicenter, randomized, double-blind placebo-controlled phase 2 study that examined the safety, tolerability, and efficacy of PZ-128 at two dose levels (0.3 and 0.5 mg/kg, versus placebo, 1:1:1), delivered intravenously in 100 patients with coronary artery disease (CAD) and acute coronary syndrome (ACS) undergoing cardiac catheterization (Kuliopulos et al., Arterioscler. Thromb. Vasc. Biol 40(12):2990-3003, 2020). PAR1 inhibitors are intended to suppress thrombin-induced platelet activation to reduce damage during percutaneous coronary intervention (PCI) and ACS, potentially reducing the risk of myocardial infarction, stroke, and serious adverse events (Mackman et al., Nat. Rev. Drug Discov. 19(5):333-352, 2020). PZ-128 clinical trial participants received a single 2-h intravenous infusion of PZ-128 or placebo started just prior to catheterization along with standard antiplatelet therapy. Overall, PZ-128 was safe and well-tolerated. Primary safety endpoints based on bleeding up to 30 days posttreatment were not significantly different between vehicle and PZ-128 dose groups, indicating that PZ-128 did not cause a significant elevation in bleeding in these patients. Secondary endpoints of major adverse coronary events (MACE) at 30 and 90 days were numerically lower in the PZ-128 groups (0% and 2% in combined PZ-128 dose groups vs 6% and 6% of placebo treated), but this trend did not reach significance (P=0.13 and 0.29, respectively), although it should be noted that this study was not powered to detect efficacy. The majority of events occurred within the first 24 h, with a nonsignificant decrease in hazard ratio (HR=0.81) of a MACE/myonecrosis event in the PZ-128 treated groups versus placebo. Interestingly, in the subgroup of patients with elevated baseline cardiac troponin I (cTnl) levels (e.g., NSTEMI patients), the exploratory endpoint of MACE and myocardial injury within 30 days was significantly reduced (P=0.02) from 83% in the placebo group to 31% in patients who received PZ-128 (Kuliopulos et al., Arterioscler. Thromb. Vasc. Biol 40(12):2990-3003, 2020). This is consistent with previous research in mouse models which indicated that PAR1 plays a role in mediating myocardial ischemia/reperfusion injury as well as cardiac remodeling and hypertrophy (Strande et al., Basic Res. Cardiol. 102(4):350-358, 2007; Sonin et al., Cardiovasc. Pharmacol. Ther. 18(5):460-475, 2013; Pawlinski et al., Circulation 116(20):2298-2306, 2007).

Analysis of ex vivo PAR1 peptide agonist (SFLLRN)-induced platelet aggregation was used to establish the pharmacodynamic properties of PZ-128 in these cardiac patients. PZ-128 effects were dose-dependent, and inhibition of SFLLRN-induced platelet aggregation could be detected by 1 h and peaked at 24 h post-dose. PAR1 inhibition by PZ-128 was reversible with higher concentrations of agonist, but long lived with 50% of the PAR1 aggregation response recovered at 14 days post-dose (Kuliopulos et al., Arterioscler. Thromb. Vasc. Biol 40(12):2990-3003, 2020). The trial confirmed that PZ-128 is a safe and efficacious inhibitor of PAR1 and identified a subset of patients undergoing cardiac catheterization for CAD and ACS—those with elevated cardiac troponins—for whom PZ-128 may reduce periprocedural myocardial necrosis thus providing support for phase 3 studies of the PZ-128 intervention in patients with more severe coronary artery disease or myocardial infarction (NSTEMI/STEMI).

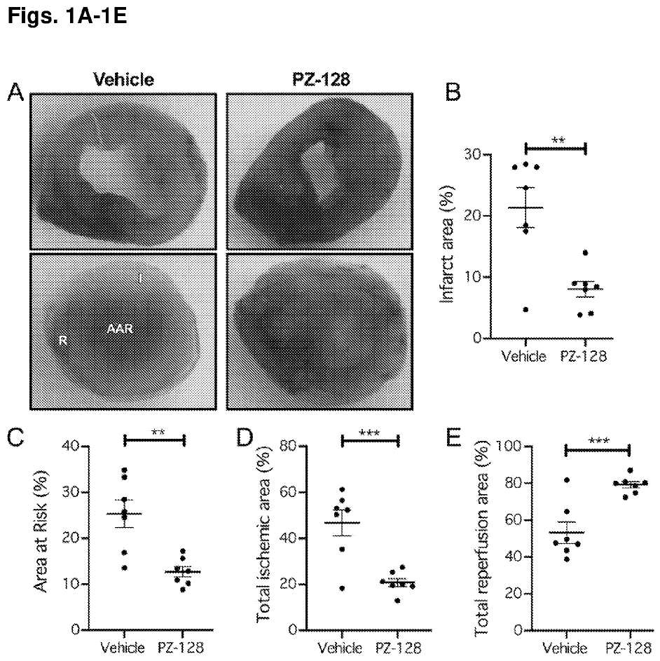

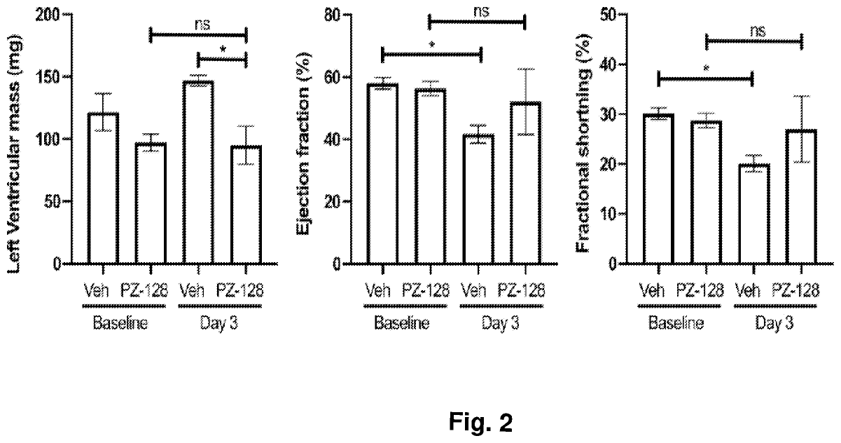

A role of PAR1 in directly mediating cardiac ischemic myonecrosis—independent of established platelet-dependent effects (Strande et al., Basic Res. Cardiol. 102(4):350-358, 2007)—was also examined. In vivo studies were conducted in mice (mice do not express PAR1 on platelets), subjected to 30 min of total occlusion of the left coronary artery and 2 h of reperfusion using published methods (Pawlinski et al., Circulation 116(20):2298-2306, 2007). Mice (n=7) were treated with vehicle or 10 mg/kg PZ-128 (subcutaneous; sc) just prior to ischemic injury. As shown in FIG. 1a-d, PZ-128-treated mice had 51-62% reductions in mean cardiac Infarct Area (p<0.01), Area At Risk (p<0.01), and Total Ischemic Area (p<0.0001) compared to vehicle-treated mice. Conversely, PZ-128-treated mice had a 50% increase in total area of cardiac muscle that was able to be reperfused (p<0.001) compared to vehicle animals (FIG. 1e). Using echocardiography after 3 days of reperfusion, there was a significant increase in left ventricular mass (p<0.05) in vehicle compared to PZ-128 treated cohorts, with no difference in left ventricular mass between baseline and 3 days in the PZ-128 cohort (FIG. 2). Likewise, after 3 days reperfusion, vehicle-treated mice had significantly lower ejection fraction and fractional shortening (p<0.05) compared to their baselines, with no significant reductions observed in the PZ-128 cohort at 3 days compared to baseline. Previous pharmacokinetic studies of subcutaneously administered PZ-128 in mice were the basis of this 10 mg/kg dose in mice (Cisowski et al., Am. J. Pathol. 179(1):513-523, 2011), selected to match the 0.3-0.5 mg/kg doses administered in humans. These new data show a direct cardioprotective function for PZ-128 beyond the antiplatelet function of PZ-128 for the suppression of myocardial necrosis that was noted during the TRIP-PCI study.

FIGS. 1A-1E show that PZ-128 protects against cardiac myocardial injury in mice. Eight-week male C57BL/6N mice were vehicle (20% DMSO) or 10 mg/kg PZ-128 1 h before cardiac surgery (n=7 per group). The left coronary artery was occluded for 30 min with a ligature left in place during reperfusion. Blood flow was restored for 2 h, then the left coronary artery was reoccluded, and 5% Evans blue dye was injected via the aorta. Hearts were frozen and sectioned in OTC, before incubation in TTC for 10 min at 37° C. (a) Sections were digitally scanned and the area of white (b—Infarct area=1), red (c—Area at Risk=AAR), white+red (d—total Ischemic area), and blue (e—Reperfusion area=R) quantified using ImageJ and adjusted by weight of each cardiac slice. Data are shown as mean±SD and analyzed by two-tailed Student's t-test: **p<0.01, ***p<0.001. These results demonstrate that PZ-128 lessens damage due to ischemia caused by reperfusion and significantly suppresses infarct area due to acute myocardial infarction, independently of platelet PAR1 as mice lack PAR1 on their platelets. Moreover, these data show that PZ-128 causes greatly increased cardiac microvascular perfusion allowing more blood to flow to the heart muscle.

FIG. 2 shows echocardiography of mouse hearts after 3 days reperfusion following 30 min myocardial ischemia treated with vehicle or 10 mg/kg PZ-128. A subset of male C57BL6N mice from each group subjected to 30 min left coronary artery ischemia (FIGS. 1A-1E) were reperfused for 3 days (n=4 vehicle, n=4 10 mg/kg PZ-128 sc once daily). Echo parameters for these mice showed a significant increase in left ventricular mass in the vehicle cohort compared to the PZ-128 group at day 3, with no significant differences for the PZ-128 group as compared to baseline. A significant decrease in both ejection fraction and fractional shortening was seen in vehicle-treated mice compared to baseline, whereas PZ-128-treated mice at day 3 maintained baseline ejection fraction and fractional shortening. Significance was determined using a repeated measures ANOVA with Bonferonni multiple test correction, *P<0.05 These results show that the ischemic heart treated acutely with PZ-128 maintains both ejection fraction and fractional shortening in the normal range, two critical parameters that indicate retention of robust left ventricular function following acute myocardial infarction as compared to the untreated cohort.

### Example 2

In a search for new targets, a genomics study showed that primary rat cardiac fibroblasts express 190 different GPCRs and that activation of PAR1, the most highly expressed of all the receptors, increased levels of profibrotic markers and collagen synthesis with conversion to a profibrogenic phenotype in tissue culture (Snead et al., Faseb J. 26(11):4540-4547, 2012). PAR1 is highly expressed in the hearts of HF patients and is associated with increased cardiac fibrosis and diastolic dysfunction (E/e′) (Friebel et al., Eur. Heart J. 40(40):3318-3332, 2019). In accordance, cardiac-overexpression of PAR1 leads to heart failure in mice (Pawlinski et al., Circulation 116(20):2298-2306, 2007). And for unknown reasons, targeting upstream of PAR1 with potent anticoagulants such as thrombin (i.e., dabigatran) or Xa (i.e., rivaroxaban or apixaban (Yurista et al., Cardiovasc. Drugs Ther. 35(5):953-963, 2021)) in HF patients has shown no clear benefit including in the large >5000 patient Commander-HF trial (Zannad et al., N. Engl. J. Med. 379(14):1332-1342, 2018). We propose that the non-canonical PAR1 agonist, MMP1 instead plays an important role in PAR1-dependent cardiac remodeling and diastolic dysfunction in HF.

The TRIP-PCI (Thrombin Receptor Inhibitory Pepducin in PCI) Phase 2 trial (Kuliopulos et al., Arterioscler. Thromb. Vasc. Biol. 40(12):2990-3003, 2020), which enrolled a subset of patients with HF, was conducted to assess the safety, target engagement, and efficacy of PZ-128 in patients undergoing cardiac catheterization with intent to perform percutaneous coronary intervention (PCI). As explained above, in this randomized, double-blind, placebo-controlled, phase 2 trial, 100 patients were randomly assigned (2:1) to receive PZ-128 (0.3 mg/kg or 0.5 mg/kg), or placebo in a two-hour infusion initiated just prior to the start of cardiac catheterization. We examined the subset of patients previously diagnosed with HF (NYHA class 1-3; ejection fractions (EF) ranging from 30% to 53%) including both HFpEF and HFrEF. Due to the small sample size, we combined both active drug groups together (0.3 mg/kg and 0.5 mg/kg PZ-128) for analysis. The baseline characteristics and hypertension/HF medications of these 9 HF patients are shown in Table 1. Patients were 47-76 years old, typically obese and had several cardiovascular disease risk factors such as hypertension (80-100%), dyslipidemia (75-100%), smoker (75-80%), diabetes (50-60%), with either angina, previous MI, PCI or CABG (50-80%). There were no statistically significant differences in baseline characteristics between placebo and PZ-128 cohorts. The PZ-128 drug was tolerated well, including in the 9 patients who had heart failure with EFs ranging from 30-53%.

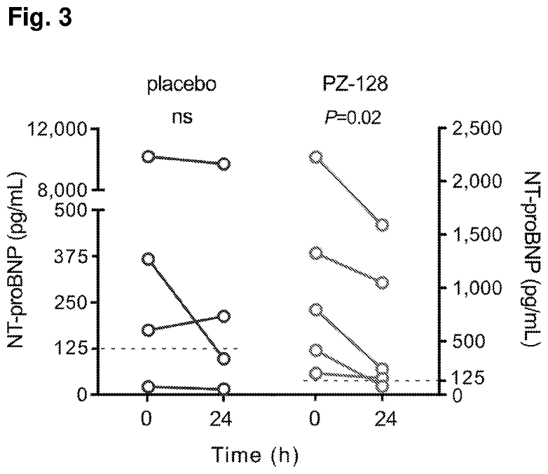

Left ventricular wall stress is the most potent trigger for release of natriuretic peptides such as BNP. The 32 amino acid polypeptide BNP is secreted attached to a 76 amino acid N-terminal fragment in the prohormone called NT-proBNP, which is biologically inactive. During the stress of HF, BNP and the N-terminal fragment NT-proBNP levels significantly increase, and correspondingly decrease with effective intervention. In the BNP multinational study of 1568 patients presenting with dyspnea to the emergency department, BNP and NT-proBNP were the single most accurate predictors of diagnosis and severity of ADHF, and are used as a well-validated surrogate clinical efficacy endpoint in human trials (Ibrahim et al., Circ. Res. 123(5):614-629, 2018; Ibrahim et al., Circ. Heart Fail. 9(9), 2016). NT-proBNP levels were measured at baseline and 24 hours after the PZ-128 or placebo infusion was initiated and the cardiac catheterization procedure completed in the subset of patients with heart failure. Baseline NT-proBNP varied with the majority of diagnosed HF patients having levels above normal (≥125 pg/mL). Those HF patients treated with a single dose of PZ-128 had a significant mean drop in NT-proBNP levels at 24 hours (P=0.02), whereas placebo had a non-significant effect (FIG. 3).

### The Effects of PZ-128 on Chronic Fibrosis and HF Parameters in the LV Pressure Overload Model

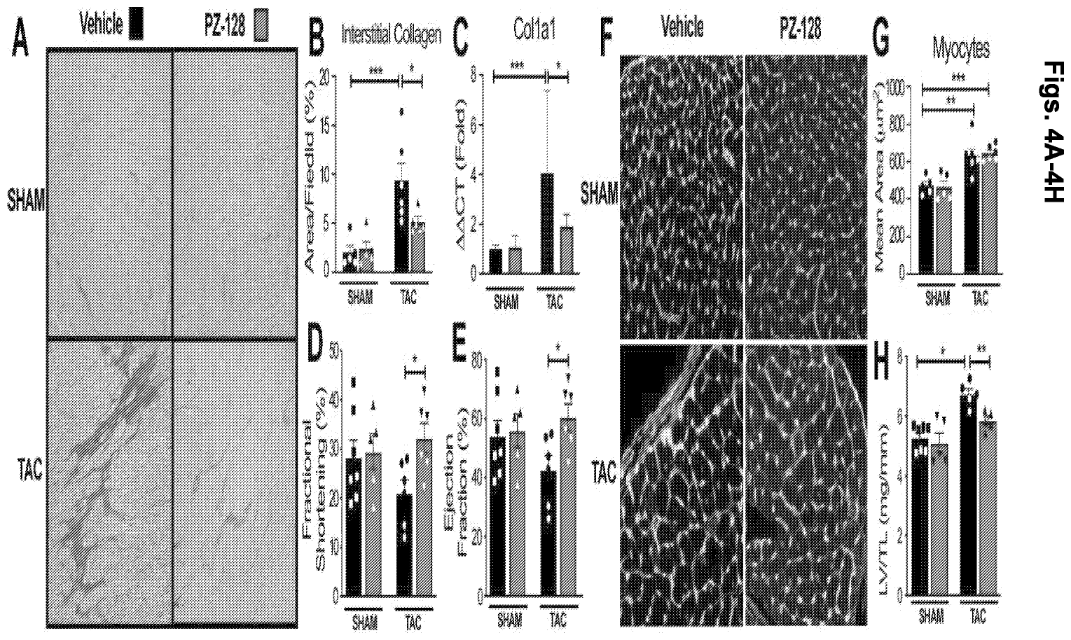

To begin to investigate more chronic PAR1-mediated mechanisms responsible for cardiac fibrosis in nonischemic HF, we adopted the mouse model of thoracic aortic constriction (TAC), which induces fibrosis and HF in response to LV pressure overload comparable to what is observed in patients with HFrEF (Nevers et al., J. Exp. Med. 214(11):3311-3329, 2017). TAC vs Sham mice were treated for 7 weeks with vehicle or PZ-128, and cardiac fibrosis and LV hypertrophy indices measured. As shown in FIGS. 4A-4C, PZ-128 gave dramatic anti-fibrotic effects on collagen and Col1A1 expression relative to vehicle in the HFrEF model. PZ-128 also gave significant protective effects of the TAC-induced losses in fractional shortening and ejection fraction by ECHO (FIGS. 4D-4E). Interestingly, despite having no effect on suppressing the increase in mean myocyte cross-sectional area, PZ-128 conferred a highly significant protective effect in LV mass (adjusted for tibia length) (FIGS. 4G-4H). This may indicate that unlike upstream thrombin (Dong et al., Thromb. Res. 159:58-64, 2017) or factor Xa (Yurista et al., Cardiovasc. Drugs Ther. 35(5):953-963, 2021) blockade, inhibition of PAR1 may effectively suppress cardiac hypertrophy by reducing the total ventricular mass, potentially by reducing MMP1-PAR1 driven fibrosis/remodeling and cardiac fibroblast expansion.

### Suppressive Effects of PAR1 Blockade on Cardiac Leukocyte Infiltration in the LV Pressure Overload Model

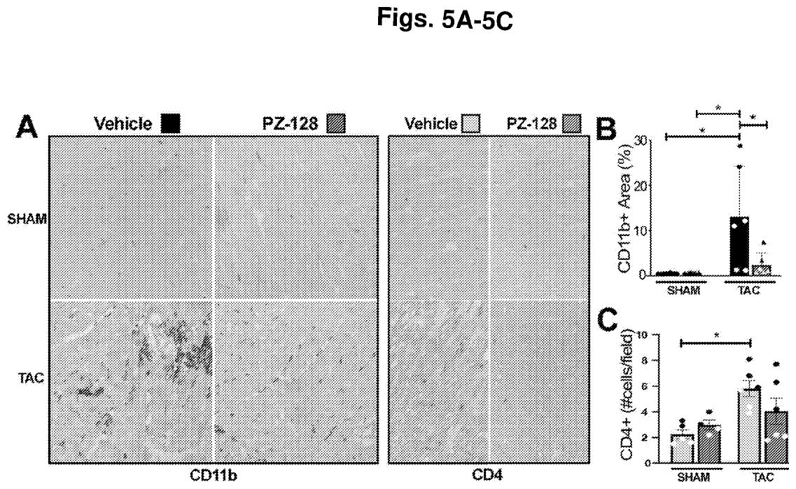

To obtain additional data to support PAR1 as a potentially important contributor to HF pathogenesis via inflammation, we investigated the effect of PZ-128 on monocyte/macrophage and CD4+ T cells in the 7-week TAC LV pressure overload model. As shown in FIGS. 5A-5B, there was a marked amount of CD11 b+monocytes/macrophages in the cardiac tissue from the TAC animals which was suppressed by nearly 90% by PZ-128 treatment. PZ-128 gave non-significant increases in T cell infiltration, compared to significant increases in vehicle mice (FIGS. 5A and 5C).

### Effects of PZ-128 on Cardiac Fibrosis and Inflammatory Gene Expression in the TAC Model.

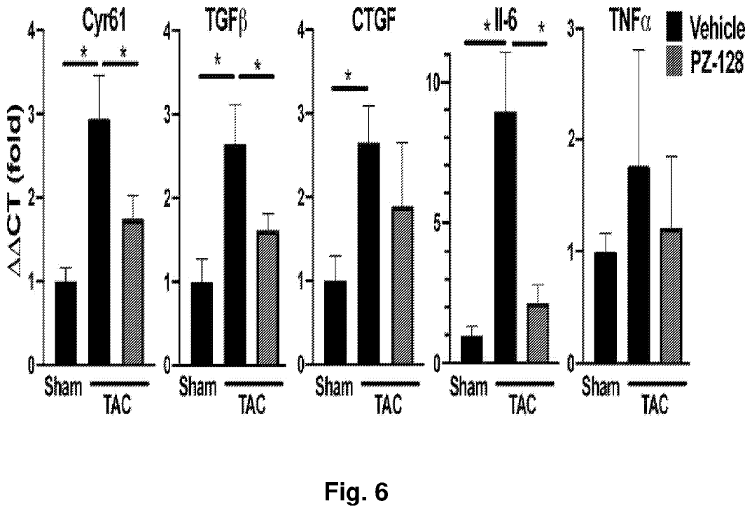

Consistent with the suppressive effects of PZ-128 on cardiac fibrosis and leukocyte infiltration, preliminary gene expression analysis indicated that the PAR1 pepducin reduces expression of key fibrotic factors Cyr61, TGFβ, and CTGF, and inflammatory cytokines IL-6 and TNFα in the TAC mice (FIG. 6).

TABLE 1
Baseline heart failure patient characteristics and medications

| | Placebo | | PZ-128 | |
|---|---|---|---|---|
| | (n = 4) | | (n = 5) | |
| Demographics | | | | |
| Age (years) | 62 ± 4 | | 62 ± 13 | |
| Range (years) | 56-66 | | 47-76 | |
| Weight (kg) | 115 ± 23 | | 107 ± 20 | |
| Body-mass index (kg/m²) | 37 ± 6\| | | 35 ± 5 | |
| Heart Failure NYHA class | 1-2 | | 1-3 | |
| Cardiovascular risk factors | | | | |
| Hypertension | 4 | (100%) | 4 | (80%) |
| Dyslipidemia | 3 | (75%) | 5 | (100%) |
| Smoker (former or current) | 3 | (75%) | 4 | (80%) |
| Diabetes | 2 | (50%) | 3 | (60%) |
| Cardiovascular disease history | | | | |
| Previous MI | 2 | (50%) | 3 | (60%) |
| Previous PCI or CABG | 2 | (50%) | 3 | (60%) |
| Angina | 2 | (50%) | 4 | (80%) |
| Blood pressure/HF medications | | | | |
| Angiotensin II receptor blocker | 1 | (25%) | 2 | (40%) |
| Angiotensin converting enzyme inhibitor | 2 | (50%) | 3 | (60%) |
| Beta Blocker | 3 | (75%) | 5 | (100%) |
| Diuretic | 2 | (50%) | 5 | (100%) |

### Other Embodiments

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Some embodiments are within the scope of the following numbered paragraphs.

1. A method of providing direct cardioprotection to a subject, the method comprising administering PZ-128 to the subject.

2. A method for preventing, reducing, or treating myocardial stress in a subject, the method comprising administering PZ-128 to the subject.

3. The method of paragraph 1 or 2, wherein the PZ-128 is administered parenterally.

4. The method of paragraph 2 or 3, wherein the myocardial stress is assessed by measurement of B-type (Brain) Natriuretic Peptide (BNP) or an N-terminal fragment thereof in a sample from the subject, and a reduction in BNP or an N-terminal fragment thereof of indicates that the treatment has resulted in prevention, reduction, or treatment of myocardial stress in the subject.

5. The method of any one of paragraphs 1 to 4, wherein the subject has or is at risk of developing heart failure (HF).

6. The method of paragraph 5, wherein the HF is HF with preserved ejection fraction (HFpEF).

7. The method of paragraph 5, wherein the HF is HF with reduced ejection fraction (HFrEF).

8. The method of paragraph 5, wherein the HF is intermediate HF.

9. The method of any one of paragraphs 1 to 8, wherein the subject has recently or is currently experiencing acute coronary syndrome (ACS).

10. The method of any one of paragraphs 1 to 9, wherein the subject has recently or is currently experiencing ST-segment-elevation myocardial infarction (STEMI).

11. The method of any one of paragraphs 1 to 10, wherein the subject has recently or is currently experiencing Non-ST-segment-elevation myocardial infarction (NSTEMI).

12. The method of paragraph any one of paragraphs 1 to 11, wherein the subject has recently or is currently experiencing unstable angina.

13. The method of any one of paragraph 1 to 12, wherein the treatment is carried out in an acute care setting within 3 days of symptom onset.

14. The method of any one of paragraphs 1 to 13, wherein the treatment is carried out for five or fewer days, for example, one to three days.

15. The method of any one of paragraphs 1 to 14, wherein the subject is administered PZ-128 once daily for three days.

16. The method of any one of paragraphs 1 to 13, wherein the subject is administered PZ-128 for 1-4 weeks or more, for example, for up to 1, 2, or 3 months.

17. The method of paragraph 16, wherein the PZ-128 is administered once daily, every 2 days, every 3 days, twice weekly, once weekly, biweekly, or monthly.

18. The method of any one of paragraphs 1 to 17, wherein the method improves ejection fraction (EF) in the subject.

19. The method of any one of paragraphs 1 to 18, wherein the method improves fractional shortening by echocardiogram in a subject.

20. The method of any one of paragraphs 1 to 19, wherein the method improves left ventricular function in the subject.

21. The method of any one of paragraphs 1 to 20, wherein the method improves coronary microvascular blood flow.

22. The method of any one of paragraphs 1 to 21, wherein the method increases reperfusion area in the heart of the subject.

23. The method of any one of paragraphs 1 to 22, wherein the method decreases myonecrosis in the heart of the subject.

24. The method of any one of paragraphs 1 to 23, wherein the method decreases infarct size in the heart of the subject.

25. The method of any one of paragraphs 1 to 24, wherein the subject has or is at risk of developing cardiac fibrosis, and the treatment prevents, reduces, or treats the cardiac fibrosis.

26. The method of any one of paragraphs 1 to 25, wherein the subject is characterized by elevated levels of cardiac troponins.

27. The method of any one of paragraphs 1 to 26, wherein the parenteral administration is intravenous administration.

28. The method of any one of paragraphs 1 to 27, wherein the subject is administered 0.1 mg/kg-1.0 mg/kg PZ-128 by 1-4 hours of intravenous infusion.

29. The method of any one of paragraphs 1 to 28, wherein the subject is administered about 0.3 or about 0.5 mg/kg PZ-128 by intravenous infusion for about 2 hours.

30. The method of any one of paragraphs 1 to 29, wherein the method further comprises thrombolytic therapy.

31. The method of any one of paragraphs 1 to 30, wherein the subject is receiving cardiac catheterization.

32. The method of any one of paragraphs 1 to 30, wherein the method further comprises percutaneous coronary intervention (PCI).

33. The method of any one of paragraphs 1 to 32, wherein the administration is initiated within 1 hour before percutaneous coronary intervention (PCI) and optionally is carried out in combination with oral antiplatelet therapy.

34. The method of any one of paragraphs 1 to 33, wherein the administration is carried out in a subject who is not treated by PCI.

35. The method of any one of paragraphs 1 to 34, wherein the subject is treated by coronary artery bypass graft (CABG) after PZ-128 treatment and is not treated by PCI.

36. The method of any one of paragraphs 1 to 35, further comprising administration of one or more drug selected from the following categories: anti-hypertensives, angiotensin receptor-neprilysin inhibitors (e.g., sacubitril/valsartan; Entresto), sodium-glucose co-transporter-2 (SGLT2) inhibitors (e.g., canagliflozin, dapagliflozin, and empagliflozin); angiotensin receptor blockers (e.g., losartan, candesartan, and telmesartan), renin-angiotensin antagonists (ARBs, ACE inhibitors (e.g., enalapril, captopril and ramipril)), beta blockers (e.g., atenolol, metoprolol, nadolol, pindolol, carvedilol, and labetelol), mineralocorticoid receptor antagonists (e.g., spironolactone, eplerenone, canrenone, finerenone, and mexrenone), diuretics (e.g., hydroclorathiazide, thiazide, indapamide, and chlorathalidone), hydralazine/nitrates (e.g., BiDil; isosorbide dinitrate and hydralazine hcl), and calcium channel blockers (e.g., amlodipine, nifedipine, nicardipine and verapamil)

37. The method of any one of paragraphs 1 to 36, further comprising treatment of the subject with a ventricular assist devise, such as IMPELLA.

38. The method of any one of paragraphs 1 to 37, wherein the subject is at high risk for bleeding.

39. The method of any one of paragraphs 1 to 38, wherein the subject is 75 years old or older.

40. The method of any one of paragraphs 1 to 39, wherein the subject is not treated with aspirin.

41. The method of any one of paragraphs 1 to 40, wherein the PZ-128 is in the form of micelles.

Some embodiments are within the scope of the following numbered paragraphs.

1. A method of providing direct cardioprotection to a subject, the method comprising administering palmitate-KKSRALF-NH₂ or a pharmaceutically acceptable salt thereof (e.g., the acetate salt) to the subject.

2. A method for preventing, reducing, or treating myocardial stress in a subject, the method comprising administering palmitate-KKSRALF-NH₂ or a pharmaceutically acceptable salt thereof (e.g., the acetate salt) to the subject.

3. The method of paragraph 1 or 2, wherein the palmitate-KKSRALF-NH₂ or a pharmaceutically acceptable salt thereof (e.g., the acetate salt) is administered parenterally.

4. The method of paragraph 2 or 3, wherein the myocardial stress is assessed by measurement of B-type (Brain) Natriuretic Peptide (BNP) or an N-terminal fragment thereof in a sample from the subject, and a reduction in BNP or an N-terminal fragment thereof of indicates that the treatment has resulted in prevention, reduction, or treatment of myocardial stress in the subject.

5. The method of any one of paragraphs 1 to 4, wherein the subject has or is at risk of developing heart failure (HF).

6. The method of paragraph 5, wherein the HF is HF with preserved ejection fraction (HFpEF).

7. The method of paragraph 5, wherein the HF is HF with reduced ejection fraction (HFrEF).

8. The method of paragraph 5, wherein the HF is intermediate HF.

9. The method of any one of paragraphs 1 to 8, wherein the subject has recently or is currently experiencing acute coronary syndrome (ACS).

10. The method of any one of paragraphs 1 to 9, wherein the subject has recently or is currently experiencing ST-segment-elevation myocardial infarction (STEMI).

11. The method of any one of paragraphs 1 to 10, wherein the subject has recently or is currently experiencing Non-ST-segment-elevation myocardial infarction (NSTEMI).

12. The method of paragraph any one of paragraphs 1 to 11, wherein the subject has recently or is currently experiencing unstable angina.

13. The method of any one of paragraph 1 to 12, wherein the treatment is carried out in an acute care setting within 3 days of symptom onset.

14. The method of any one of paragraphs 1 to 13, wherein the treatment is carried out for five or fewer days, for example, one to three days.

15. The method of any one of paragraphs 1 to 14, wherein the subject is administered palmitate-KKSRALF-NH₂ or a pharmaceutically acceptable salt thereof (e.g., the acetate salt) once daily for three days.

16. The method of any one of paragraphs 1 to 13, wherein the subject is administered palmitate-KKSRALF-NH₂ or a pharmaceutically acceptable salt thereof (e.g., the acetate salt) for 1-4 weeks or more, for example, for up to 1, 2, or 3 months.

17. The method of paragraph 16, wherein the palmitate-KKSRALF-NH₂ or a pharmaceutically acceptable salt thereof (e.g., the acetate salt) is administered once daily, every 2 days, every 3 days, twice weekly, once weekly, biweekly, or monthly.

18. The method of any one of paragraphs 1 to 17, wherein the method improves ejection fraction (EF) in the subject.

19. The method of any one of paragraphs 1 to 18, wherein the method improves fractional shortening by echocardiogram in a subject.

20. The method of any one of paragraphs 1 to 19, wherein the method improves left ventricular function in the subject.

21. The method of any one of paragraphs 1 to 20, wherein the method improves coronary microvascular blood flow.

22. The method of any one of paragraphs 1 to 21, wherein the method increases reperfusion area in the heart of the subject.

23. The method of any one of paragraphs 1 to 22, wherein the method decreases myonecrosis in the heart of the subject.

24. The method of any one of paragraphs 1 to 23, wherein the method decreases infarct size in the heart of the subject.

25. The method of any one of paragraphs 1 to 24, wherein the subject has or is at risk of developing cardiac fibrosis, and the treatment prevents, reduces, or treats the cardiac fibrosis.

26. The method of any one of paragraphs 1 to 25, wherein the subject is characterized by elevated levels of cardiac troponins.

27. The method of any one of paragraphs 1 to 26, wherein the parenteral administration is intravenous administration.

28. The method of any one of paragraphs 1 to 27, wherein the subject is administered 0.1 mg/kg-1.0 mg/kg palmitate-KKSRALF-NH₂ or a pharmaceutically acceptable salt thereof (e.g., the acetate salt) by 1-4 hours of intravenous infusion.

29. The method of any one of paragraphs 1 to 28, wherein the subject is administered about 0.3 or about 0.5 mg/kg palmitate-KKSRALF-NH₂ or a pharmaceutically acceptable salt thereof (e.g., the acetate salt) by intravenous infusion for about 2 hours.

30. The method of any one of paragraphs 1 to 29, wherein the method further comprises thrombolytic therapy.

31. The method of any one of paragraphs 1 to 30, wherein the subject is receiving cardiac catheterization.

32. The method of any one of paragraphs 1 to 30, wherein the method further comprises percutaneous coronary intervention (PCI).

33. The method of any one of paragraphs 1 to 32, wherein the administration is initiated within 1 hour before percutaneous coronary intervention (PCI) and optionally is carried out in combination with oral antiplatelet therapy.

34. The method of any one of paragraphs 1 to 33, wherein the administration is carried out in a subject who is not treated by PCI.

35. The method of any one of paragraphs 1 to 34, wherein the subject is treated by coronary artery bypass graft (CABG) after palmitate-KKSRALF-NH₂ or a pharmaceutically acceptable salt thereof (e.g., the acetate salt) treatment and is not treated by PCI.

36. The method of any one of paragraphs 1 to 35, further comprising administration of one or more drug selected from the following categories: anti-hypertensives, angiotensin receptor-neprilysin inhibitors (e.g., sacubitril/valsartan; Entresto), sodium-glucose co-transporter-2 (SGLT2) inhibitors (e.g., canagliflozin, dapagliflozin, and empagliflozin); angiotensin receptor blockers (e.g., losartan, candesartan, and telmesartan), renin-angiotensin antagonists (ARBs, ACE inhibitors (e.g., enalapril, captopril and ramipril)), beta blockers (e.g., atenolol, metoprolol, nadolol, pindolol, carvedilol, and labetelol), mineralocorticoid receptor antagonists (e.g., spironolactone, eplerenone, canrenone, finerenone, and mexrenone), diuretics (e.g., hydroclorathiazide, thiazide, indapamide, and chlorathalidone), hydralazine/nitrates (e.g., BiDil; isosorbide dinitrate and hydralazine hcl), and calcium channel blockers (e.g., amlodipine, nifedipine, nicardipine and verapamil) 37. The method of any one of paragraphs 1 to 36, further comprising treatment of the subject with a ventricular assist devise, such as IMPELLA.

38. The method of any one of paragraphs 1 to 37, wherein the subject is at high risk for bleeding.

39. The method of any one of paragraphs 1 to 38, wherein the subject is 75 years old or older.

40. The method of any one of paragraphs 1 to 39, wherein the subject is not treated with aspirin.

41. The method of any one of paragraphs 1 to 40, wherein the compound administered is palmitate-KKSRALF-NH₂ (PK-128).

42. The method of any one of paragraphs 1 to 41, wherein the compound administered is a pharmaceutically acceptable salt of palmitate-KKSRALF-NH₂ (PK-128).

43. The method of any one of paragraphs 1 to 42, wherein the compound administered is the acetate salt of palmitate-KKSRALF-NH₂ (PK-128).

44. The method of any one of paragraphs 1 to 43, wherein the compound is the palmitate KKSRALF-NH₂, or pharmaceutically acceptable salt thereof, is in the form of micelles.

Other embodiments are within the scope of the claims.

What is claimed is:

1. A method of providing direct cardioprotection to a subject, the method comprising administering PZ-128 to the subject.

2. A method for preventing, reducing, or treating myocardial stress in a subject, the method comprising administering PZ-128 to the subject.

3. The method of claim 1 or 2, wherein the PZ-128 is administered parenterally.

4. The method of claim 2, wherein the myocardial stress is assessed by measurement of B-type (Brain) Natriuretic Peptide (BNP) or an N-terminal fragment thereof in a sample from the subject, and a reduction in BNP or an N-terminal fragment thereof of indicates that the treatment has resulted in prevention, reduction, or treatment of myocardial stress in the subject.

5. The method claim 1 or 2, wherein the subject has or is at risk of developing heart failure (HF).

6. The method of claim 5, wherein the HF is HF with preserved ejection fraction (HFpEF).

7. The method of claim 5, wherein the HF is HF with reduced ejection fraction (HFrEF).

8. The method of claim 5, wherein the HF is intermediate HF.

9. The method of claim 1 or 2, wherein the subject has recently or is currently experiencing acute coronary syndrome (ACS).

10. The method of claim 1 or 2, wherein the subject has recently or is currently experiencing ST-segment-elevation myocardial infarction (STEMI).

11. The method of claim 1 or 2, wherein the subject has recently or is currently experiencing Non-ST-segment-elevation myocardial infarction (NSTEMI).

12. The method of claim 1 or 2, wherein the subject has recently or is currently experiencing unstable angina.

13. The method of claim 1 or 2, wherein the treatment is carried out in an acute care setting within 3 days of symptom onset.

14. The method of claim 1 or 2, wherein the treatment is carried out for five or fewer days, for example, one to three days.

15. The method of claim 1 or 2, wherein the subject is administered PZ-128 once daily for three days.

16. The method of claim 1 or 2, wherein the subject is administered PZ-128 for 1-4 weeks or more, for example, for up to 1, 2, or 3 months.

17. The method of claim 16, wherein the PZ-128 is administered once daily, every 2 days, every 3 days, twice weekly, once weekly, biweekly, or monthly.

18. The method of claim 1 or 2, wherein the method improves ejection fraction (EF) in the subject.

19. The method of claim 1 or 2, wherein the method improves fractional shortening by echocardiogram in a subject.

20. The method of claim 1 or 2, wherein the method improves left ventricular function in the subject.

21. The method of claim 1 or 2, wherein the method improves coronary microvascular blood flow.

22. The method of claim 1 or 2, wherein the method increases reperfusion area in the heart of the subject.

23. The method of claim 1 or 2, wherein the method decreases myonecrosis in the heart of the subject.

24. The method of claim 1 or 2, wherein the method decreases infarct size in the heart of the subject.

25. The method of claim 1 or 2, wherein the subject has or is at risk of developing cardiac fibrosis, and the treatment prevents, reduces, or treats the cardiac fibrosis.

26. The method of claim 1 or 2, wherein the subject is characterized by elevated levels of cardiac troponins.

27. The method of claim 1 or 2, wherein the parenteral administration is intravenous administration.

28. The method of claim 1 or 2, wherein the subject is administered 0.1 mg/kg-1.0 mg/kg PZ-128 by 1-4 hours of intravenous infusion.

29. The method of claim 1 or 2, wherein the subject is administered about 0.3 or about 0.5 mg/kg PZ-128 by intravenous infusion for about 2 hours.

30. The method of claim 1 or 2, wherein the method further comprises thrombolytic therapy.

31. The method of claim 1 or 2, wherein the subject is receiving cardiac catheterization.

32. The method of claim 1 or 2, wherein the method further comprises percutaneous coronary intervention (PCI).

33. The method of claim 1 or 2, wherein the administration is initiated within 1 hour before percutaneous coronary intervention (PCI) and optionally is carried out in combination with oral antiplatelet therapy.

34. The method of claim 1 or 2, wherein the administration is carried out in a subject who is not treated by PCI.

35. The method of claim 1 or 2, wherein the subject is treated by coronary artery bypass graft (CABG) after PZ-128 treatment and is not treated by PCI.

36. The method of claim 1 or 2, further comprising administration of one or more drug selected from the following categories: anti-hypertensives, angiotensin receptor-neprilysin inhibitors (e.g., sacubitril/valsartan; Entresto), sodium-glucose co-transporter-2 (SGLT2) inhibitors (e.g., canagliflozin, dapagliflozin, and empagliflozin); angiotensin receptor blockers (e.g., losartan, candesartan, and telmesartan), renin-angiotensin antagonists (ARBs, ACE inhibitors (e.g., enalapril, captopril and ramipril)), beta blockers (e.g., atenolol, metoprolol, nadolol, pindolol, carvedilol, and labetelol), mineralocorticoid receptor antagonists (e.g., spironolactone, eplerenone, canrenone, finerenone, and mexrenone), diuretics (e.g., hydroclorathiazide, thiazide, indapamide, and chlorathalidone), hydralazine/nitrates (e.g., BiDil; isosorbide dinitrate and hydralazine hcl), and calcium channel blockers (e.g., amlodipine, nifedipine, nicardipine and verapamil)

37. The method of claim 1 or 2, further comprising treatment of the subject with a ventricular assist devise, such as IMPELLA.

38. The method of claim 1 or 2, wherein the subject is at high risk for bleeding.

39. The method of claim 1 or 2, wherein the subject is 75 years old or older.

40. The method of claim 1 or 2, wherein the subject is not treated with aspirin.

41. The method of claim 1 or 2, wherein the PK-128 is in the form of micelles.
